(19) United States
(12) Patent Application Publication
(10) Pub. No.: US 2023/0293873 A1
(43) Pub. Date: Sep. 21, 2023

(54) Handheld microneedling device for local stimulation of a skin, skin stimulation device and articles

(71) Applicant: MT.DERM GmbH, Berlin (DE)

(72) Inventor: Dirk Scherkowski, Berlin (DE)

(21) Appl. No.: 18/184,820

(22) Filed: Mar. 16, 2023

(30) Foreign Application Priority Data
Mar. 16, 2022 (EP) 22162404.2

(51) Int. Cl. A61M 37/00

(52) U.S. Cl. CPC A61M 37/0015; A61M 2037/0046

(57) ABSTRACT

A handheld microneedling device for local stimulation of a skin includes: a housing; a drive device within the housing and adapted to provide a driving force; a skin stimulation device; a microneedling stimulation tool formed on the skin stimulation device and connected to the drive device such that the microneedling stimulation tool is movable back and forth at a repetition frequency by means of the driving force in operation; and a needle plate which is formed on the microneedling stimulation tool and in which a plurality of non-piercing stimulation needles with a blunt needle tip are arranged distributed over a front-side application surface. The microneedling stimulation tool is movable back and forth at the repetition frequency between a front operating position and a rear operating position which is set back in comparison thereto. Furthermore, a skin stimulation device and an article are created.

The invention relates to a handheld microneedling device for locally stimulating a skin, a skin stimulation device for a handheld microneedling device, and an article.

### BACKGROUND

Microneedling may provide for piercing the skin locally with multiple piercing needles in a planar skin section at the same time. The multiple needles are arranged on a micro needling skin piercing tool of a handheld device distributed over an application area, for example on a needle plate.

Microneedling, due to the local piercing, causes minute injuries to the skin, which the skin registers as wounds. Various messenger substances and growth factors are then released in the skin to stimulate wound healing. This process also stimulates the production of collagen, elastin and hyaluronic acid, which promote skin tightening and elasticity. Microneedling can be performed with or without the introduction of substances or active ingredients into the skin. The skin is locally pierced to trigger positive healing reactions, which can be optionally supported by substance insertion.

For the application of microneedling, for example, manual skin piercing devices of a stamp-like design are known, in which the microneedling skin piercing tool is formed with a stamp, in which several piercing needles are arranged distributed over an application surface. In other manual skin piercing devices, the piercing needles are distributed over the lateral surface of a roller, which is rolled over the skin for local piercing.

Furthermore, handheld microneedling devices in the form of a so-called pen are known, in which a microneedling skin piercing tool of a skin piercing device is formed with a needle plate, on which the several piercing needles are arranged in the area of an application surface. The microneedling skin piercing tool is automatically moved back and forth by means of an electric motor of a drive device. The electric motor of the drive unit provides a driving force that is coupled to the microneedling skin piercing tool in order to move the needle plate with the application surface and the piercing needles distributed over the surface back and forth.

A handheld microneedling device is known, for example, from the document US 2017 /0354810 A1.

### SUMMARY

It is an object of the invention to provide a handheld microneedling device for local stimulation and/or regeneration of a skin, which enables an extended application of microneedling.

The task is solved by a handheld microneedling device for local stimulation and/or regeneration of a skin according to claim 1. Furthermore, a skin stimulation device for a handheld microneedling device as well as an article according to the subordinate claims 15 and 16 are provided. Embodiments are the subject of dependent subclaims.

According to one aspect, a handheld microneedling device for locally stimulating a skin is provided, comprising: a housing; a drive device disposed in the housing and adapted to provide a driving force; a skin stimulation device disposed in the housing; a microneedling stimulation tool formed on the skin stimulation device and connected to the drive device such that the microneedling stimulation tool is movable back and forth at a repetition frequency by means of the driving force in operation; and a needle plate which is formed on the microneedling stimulation tool and in which a plurality of non-piercing stimulation needles having a blunt needle tip are arranged distributed over a front side application surface. The microneedling stimulation tool is movable back and forth at the repetition frequency between a front working position and a rear working position set back in comparison thereto by means of the driving force during operation.

According to a further aspect, there is provided a skin stimulation device having a housing and being couplable to a drive device for forming a handheld microneedling device, comprising: a microneedling stimulation tool formed on the skin stimulation device and connectable to the drive device, such that the microneedling stimulation tool is movable back and forth at a repetition frequency in operation by means of a driving force provided by the drive device; and a needle plate formed on the microneedling stimulation tool and having a plurality of non-piercing stimulation needles with a respective blunt needle tip which are arranged distributed over a front side application surface. The microneedling stimulation tool is movable back and forth between a front working position and a rear working position which is set back in comparison thereto, wherein at least the plurality of non-piercing stimulation needles of the needle plate are arranged completely outside the housing and thus exposed in the front working position and the rear working position of the microneedling stimulation tool.

According to a further aspect, an article is provided comprising: a drive device disposed in a housing selectively configured to form a handpiece; a toothbrush head releasably connectable to the housing so as to couple to the drive device to form an electric toothbrush; and a skin stimulation device releasably connectable to the housing so as to couple to the drive device to form a handheld microneedling device.

The microneedling stimulation tool can be moved back and forth along a direction of movement by means of the driving force during operation at the repetition frequency. Along the direction of movement, a movement stroke is executed between positions of the microneedling stimulation tool.

With the help of the non-piercing design of the needles, a handheld device is provided which enables a microneedling treatment (microneedling) of the skin without piercing it locally, in particular for a microneedling or a microneedling-like treatment in skin areas in which the upper skin layer (epidermis) is thinner or finer than in other skin areas, for example in the area of skin sections in the face, for example around the eyes.

The non-piercing stimulation needles can be aligned upright on the needle plate.

When moving between the front working position and the rear working position, in one embodiment, at least the plurality of non-piercing stimulation needles of the needle plate in the front working position and the rear working position of the microneedling stimulation tool may be arranged completely outside the housing and thus exposed. In an exemplary embodiment, the handheld microneedling device is structurally configured to enable further improved microneedling. Since at least the plurality of non-piercing stimulation needles of the needle plate on the microneedling stimulation tool, both in the front working position and in the rear working position, are each arranged completely outside the optionally single-piece or multipiece housing, in particular outside a housing component of the skin stimulation device, and thus exposed, the skin section to be locally stimulated can itself be set into oscillation due to the repetitive movement of the needle plate with the multiple non-piercing stimulation needles, and is therefore not prevented from doing so by the housing of the handheld microneedling device, which enables the multiple non-piercing stimulation needles to be applied efficiently and as painlessly as possible to the skin surface without penetrating the skin.

In operation, in an exemplary embodiment, at least the front-side application surface can furthermore be arranged completely outside the housing and thus exposed, in particular outside a housing interior at least partially surrounded by housing sections of the housing, both in the front working position and in the retracted rear working position of the microneedling stimulation tool. In this way, the handheld microneedling device is additionally structurally set up to permit free oscillation of the processed skin section during operation during microneedling.

The front-side application surface with the plurality of non-piercing stimulation needles distributed thereon may be slanted or inclined, at least in the front operating position, with respect to at least one reference direction selected from the group consisting of: the longitudinal direction of the housing, the direction of movement of the microneedling stimulation tool when displaced between the front and retracted rear operating positions, and the direction of movement of the needle plate when displaced between the front and rear retracted operating positions.

The front-side application surface with the several non-piercing stimulation needles distributed thereon can be inclined both in the front working position and in the rear working position, which is set back in comparison. In this case, the inclined or tilted position can also be formed during movement between the front and rear working positions.

For example, the front-side application surface may include an angle between about 30 degrees and < 90 degrees with the longitudinal direction of the housing, preferably between about 30 degrees and about 85 degrees, and further preferably between about 45 degrees and about 80 degrees.

In the case of the handheld microneedling device, a protective or cover cap may be provided which is selectively releasably arranged on the housing and covers the needle plate, a functional tool being arranged on the outside of the protective cap. For example, a plurality of skin massage projections may be arranged on the exterior of the protective cap, particularly in the region of a front surface, the skin massage projections being arranged in a distributed manner on the exterior surface of the protective cap. For example, the skin massage protrusions may have a rounded head portion, such as having a spherical head.

A functional tool is thus arranged on the cover cap, which in the fitted state covers the several non-piercing stimulation needles, which can be formed with an arrangement of functional elements, which are, for example, massage elements. The cover cap can thus be implemented as a multifunctional cap.

Openings can be provided on the cover cap. For example, the openings are arranged in the region of an outer surface of the cover cap and thus form lateral openings. Alternatively or additionally, such openings may be arranged in the region of the front surface in other embodiments. For example, the openings may serve to perform gas sterilization for the needle plate with the plurality of stimulation needles when the cover cap is in place. Side openings have the advantage of reducing the entry of dust particles to the needle plate and dust deposits caused thereby.

In alternative embodiments, the microneedling stimulation tool may have the needle plate formed free of a housing portion of the housing that at least partially surrounds the needle plate.

The needle plate can be arranged on a housing section of the housing, which is moved along with the microneedling stimulation tool when it is moved back and forth during operation. This may be a front housing section of the housing. Here, the needle plate may be formed on an outer surface of the housing portion with which the application surface is provided. The needle plate may be formed on a front housing portion of the housing, which is moved back and forth in operation together with the microneedling stimulation tool. For example, the needle plate may be integrally formed as a front plate of the housing section. The needle plate may be selectively releasably (detachably) or non-detachably disposed on the housing section.

The needle plate may be integrally molded to the housing section of the housing. In this case, a lateral wall portion of the housing portion may be integrally formed with a front plate providing the application surface. The housing section may thus be configured to form the microneedling stimulation tool, in particular the needle plate.

The microneedling stimulation tool may be detachable or removable from the housing together with the housing section. The microneedling stimulation tool and/or the skin stimulation device with the microneedling stimulation tool, whether separately or together with the housing section, may be selectively releasably mounted by means of a plug-in connection or a screw connection. Thus, the microneedling stimulation tool can be mounted together with the housing section on an associated housing section, in particular a housing section that accommodates the drive device.

In various embodiments, the plurality of stimulation needles may be applied to the needle plate in the area of the application surface. Alternatively, it may be provided that the stimulation needles are arranged in openings of the needle plate, for example soldered or glued in place, wherein the stimulation needles may extend through the openings at least in sections. It may be provided that, after passing through the openings, the stimulation needles are received on a needle holder element which is rearward in relation to the application surface of the needle plate and which may be formed as part of the microneedling stimulation tool or the needle plate.

Alternatively, the stimulation needles can be integrally formed on the needle plate. For example, the stimulation needles can be formed by means of stamped and bent sheet metal elements.

Stimulation needles in the sense of the present disclosure are generally non-sharp or non-piercing volume elements protruding from the needle plate, which in terms of shape and strength are set up for repeated (non-piercing) stimulation of the skin (layers). The stimulation needles are preferably made of a solid material, as opposed to a cannula (hollow needle).

A front housing part, which is associated with the skin stimulation device, can be formed in a sleeve-like shape at the rear and at least partially surround or enclose a rear housing part, which is associated with the drive device. In this or other embodiments, a housing component of the skin stimulation device may be formed with the front housing part, which may be removable from the rear housing part, for example a module housing of a stimulation module.

The protective cap can be selectively releasably attached to the front end of the housing.

The skin stimulation device can be selectively releasably arranged on the housing. In this way, the microneedling stimulation tool with an associated housing part can be exchangeably arranged on the housing.

The drive device can be formed with a drive device for an electric toothbrush, which is set up for selective releasable coupling thereto of the skin stimulation device and a toothbrush head which can be operated by means of the drive device. Drive devices for electric toothbrushes are known as such in various designs. The housing of the drive device often forms a handpiece of the electric toothbrush, which the user grips during use. The drive device provides a driving force/movement for the electrical operation of the toothbrush head. The toothbrush head is removable. The drive device of the electric toothbrush can then be used to form the handheld microneedling device by selectively releasably attaching the skin stimulation device to the drive device of the electric toothbrush so that the provided driving force can be introduced to the microneedling stimulation tool such that the microneedling stimulation tool is moved back and forth at a repetitive frequency by means of the driving force during operation.

The microneedling stimulation tool may be displaceable through an opening at a front end of the housing to a non-working position that is further retracted relative to the retracted rear working position, in which at least the front-side application surface of the needle plate is retracted relative to the opening. In this embodiment, the microneedling stimulation tool may be displaced to the retracted non-working position when not in use, for example, such that the plurality of stimulation needles are positioned behind an opening surface of the (housing) opening at the front end of the housing and are thus protected by means of the housing.

The housing can partially or completely surround the opening. In the retracted non-working position, the needle plate can be partially or completely surrounded by the housing so that the needle plate is then arranged within the housing interior.

During displacement through the (housing) opening, the needle plate can be spaced on one or more sides from an edge surrounding the opening. In this embodiment, the needle plate is displaced through the opening without contact in the distance areas formed on one or more sides. In particular, there is thus no guidance of the needle plate on the housing in the spacer regions. The spacing between the needle plate and the surrounding housing can be formed continuously around the needle plate.

In the further retracted non-working position, the needle plate can be arranged so as to be exposed at a distance from housing sections of the housing surrounding the needle plate. Here, the needle plate is arranged without contact in the further retracted non-working position.

The handheld microneedling device may be formed with a reusable drive module in which the drive device is disposed, and a disposable module which is releasably coupled to the drive module and in which the skin stimulation device is disposed. The drive module and the disposable module may be formed with a respective associated module housing, wherein the module housings may be releasably coupled or connected to each other.

The (in particular motorized) drive device can be set up to move the microneedling stimulation tool back and forth during operation by means of the driving force applied thereto at a repetition frequency between about 10 Hz and about 200 Hz, alternatively at a repetition frequency between about 25 Hz and about 160 Hz.

It can be provided that a distance (working stroke length) of about 0.5 mm to about 5 mm is formed between the front working position and the rear working position set back in comparison thereto in a direction of movement of the microneedling stimulation tool and/or longitudinal direction of the handheld microneedling device (in particular its housing). Alternatively, a distance (working stroke length) of only about 1 mm to about 4 mm can be formed between the front working position and the rear working position, which is set back in comparison, in the direction of movement of the microneedling stimulation tool and/or longitudinal direction of the microneedling hand held device. By means of different stroke lengths, the handheld microneedling device can be configured for different applications, for example for medical or cosmetic microneedling.

In one embodiment, the needle plate can be designed with a reversible plate or a reversible insert in such a way that different microneedling stimulation tools can be arranged on it in an exchangeable or detachable manner in order to provide different (microneedling) functions and/or to compensate for wear of the stimulation tool. They can also be interchangeable inserts that are selectively releasably arranged on the needle plate depending on the desired application. Reversible inserts can carry stimulation tools on both sides. The interchangeable inserts may be selectable from an assortment or a graduated set of tool characteristics.

The application surface on which the plurality of non-piercing stimulation needles are arranged may have a convexly curved surface that covers all or only part of the application surface, particularly only in a central surface region.

The preceding embodiments explained in connection with the handheld microneedling device may be provided in connection with the skin stimulation device and/or article accordingly.

### DESCRIPTION OF EMBODIMENT EXAMPLES

In the following, further embodiments are explained with reference to figures of a drawing. Here show:

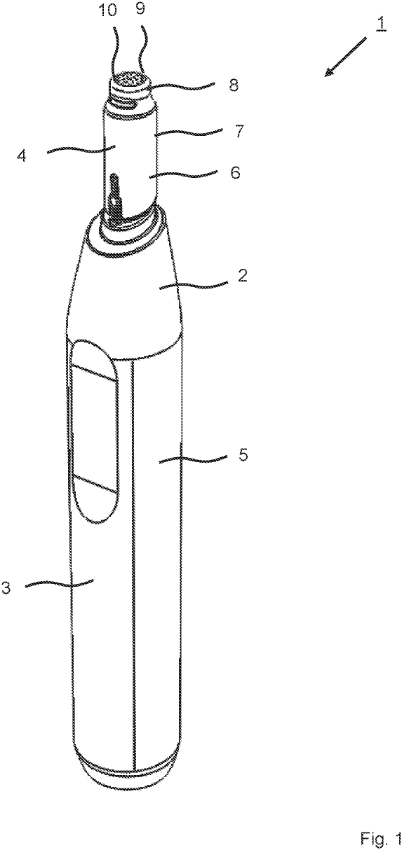

FIG. 1 a schematic perspective view of a handheld microneedling device;

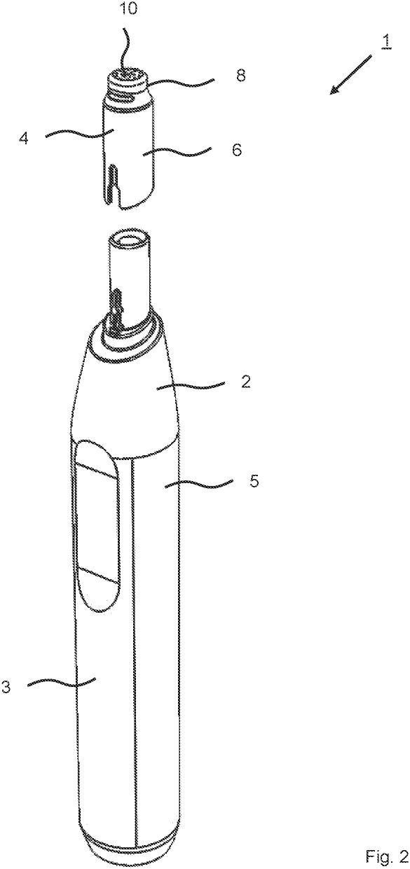

FIG. 2 a schematic perspective view of the handheld microneedling device of FIG. 1. wherein a stimulation module with a microneedling stimulation tool is detached;

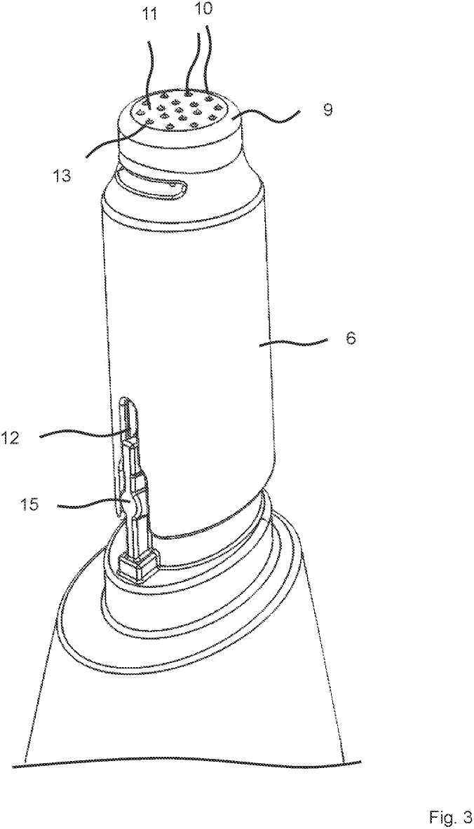

FIG. 3 a schematic perspective view of a front section of the handheld microneedling of FIG. 1, wherein the microneedling stimulation tool with needle plate is arranged in a front working position;

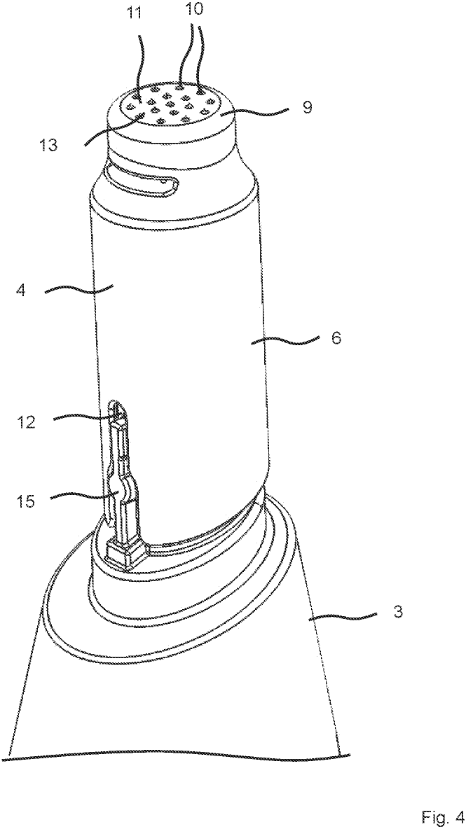

FIG. 4 a schematic perspective view of the front section of the Handheld microneedling, with the microneedling stimulation tool with needle plate arranged in a retracted rear working position;

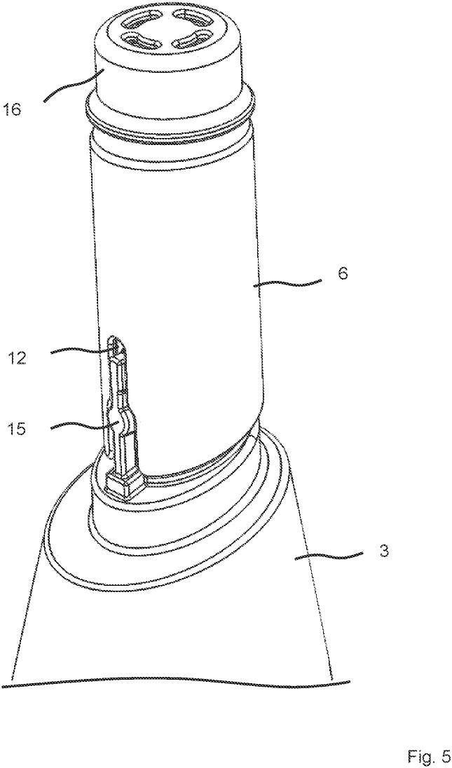

FIG. 5 a schematic perspective view of the front section of the micro-needling handpiece, wherein a cover or protective cap is placed on the front of the microneedling stimulation device covering the needle plate;

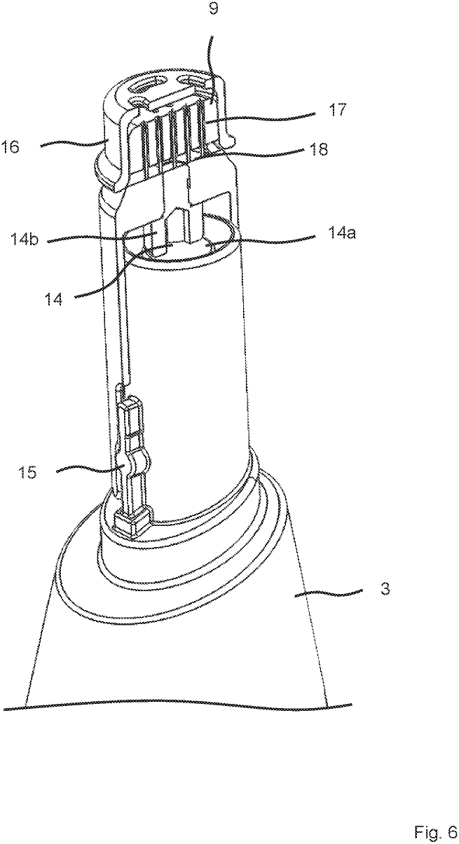

FIG. 6 a schematic perspective view of the front section of the handheld microneedling device partially in section with the cover or protective cap in place;

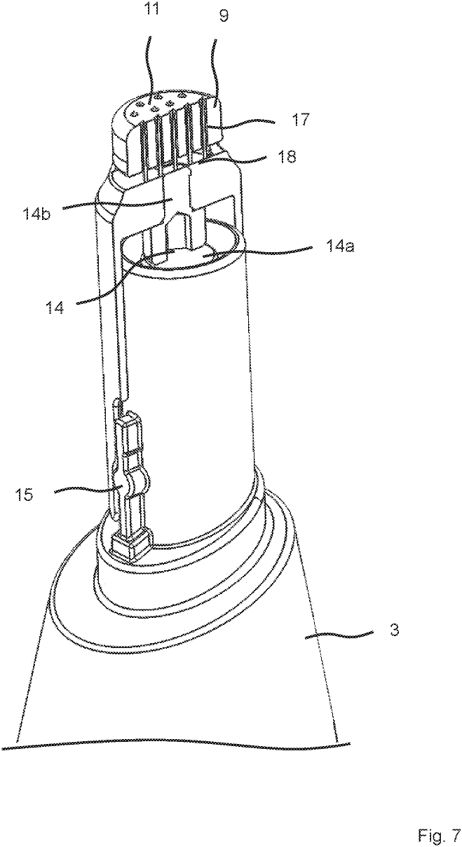

FIG. 7 a schematic perspective view of the front section of the Handheld microneedling partially in section with the cover or protective cap removed;

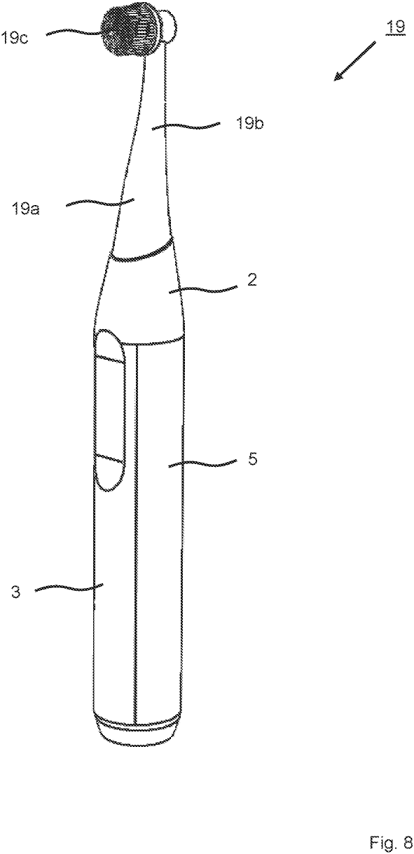

FIG. 8 a schematic perspective view of an electric toothbrush;

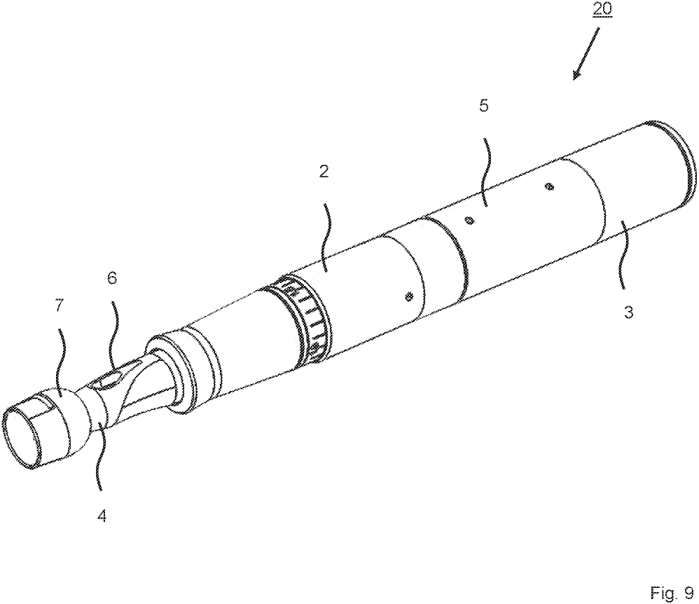

FIG. 9 a schematic perspective view of another handheld microneedling device;

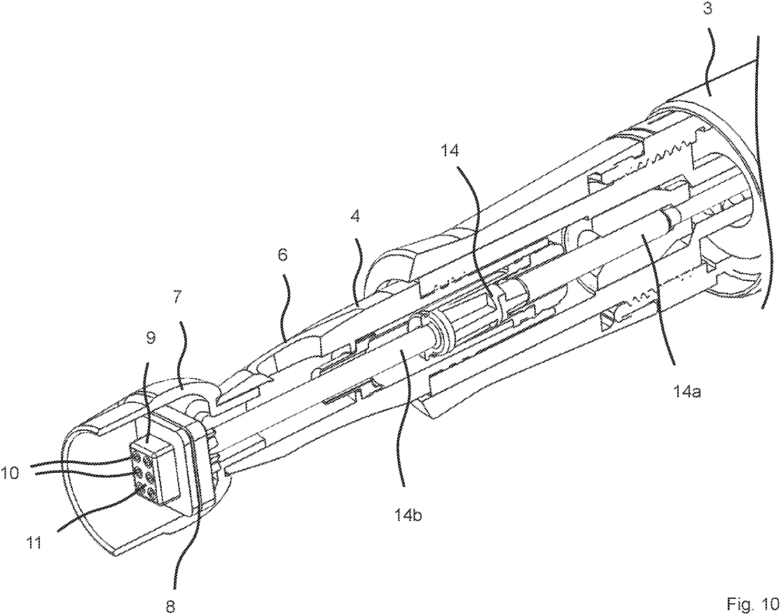

FIG. 10 a schematic perspective view of a front housing part of the handheld microneedling device of FIG. 9 in section;

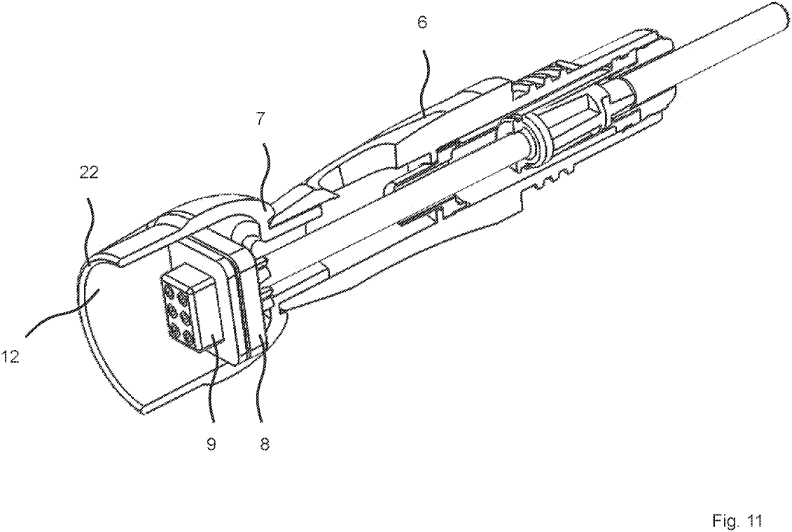

FIG. 11 a schematic perspective view of the front housing part of the handheld microneedling device of FIG. 9, with the needle plate of the microneedling stimulation tool arranged in a retracted non-working position;

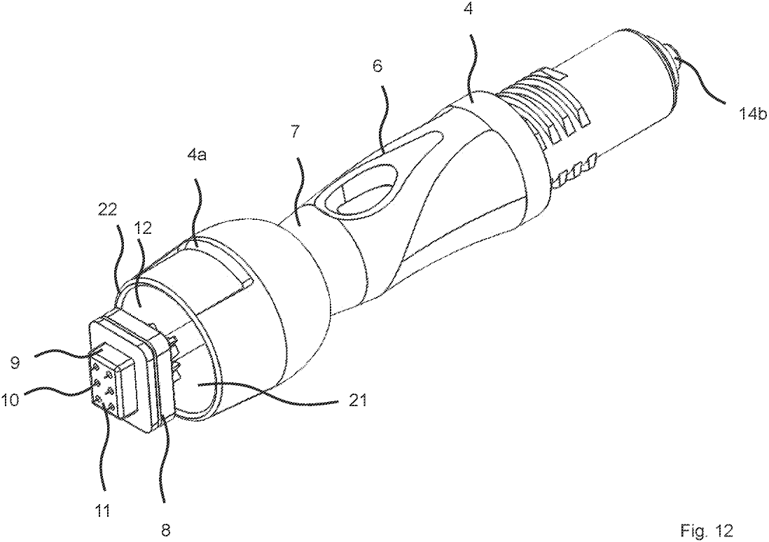

FIG. 12 a schematic perspective view of the front housing part of the handheld microneedling device of FIG. 9, with the needle plate of the microneedling stimulation tool exposed in a front working position;

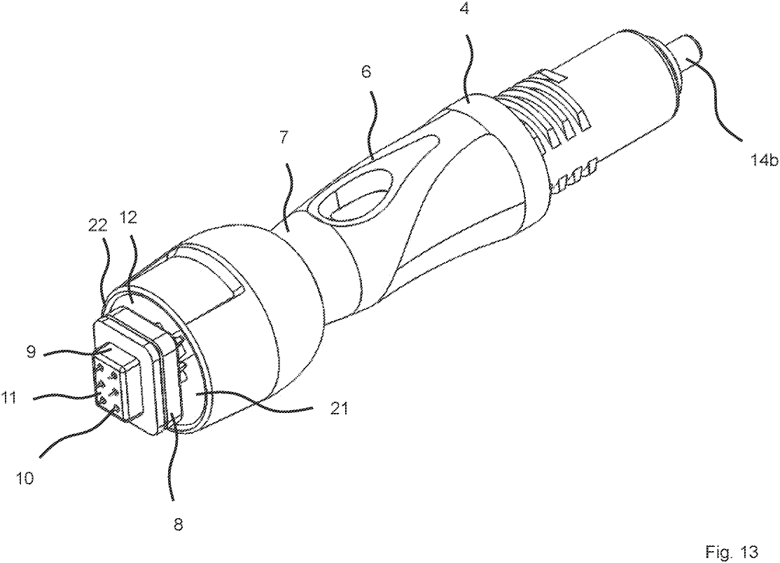

FIG. 13 a schematic perspective view of the front housing part of the handheld microneedling device from FIG. 9, with the needle plate exposed in a retracted rear working position;

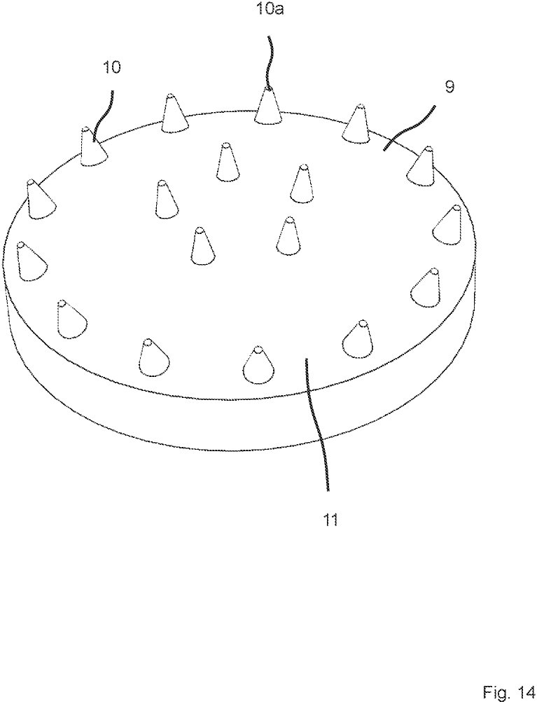

FIG. 14 a schematic perspective view of a needle plate with an arrangement of non-piercing stimulation needles with blunt needle tip in the area of a frontal application surface, seen from diagonally above;

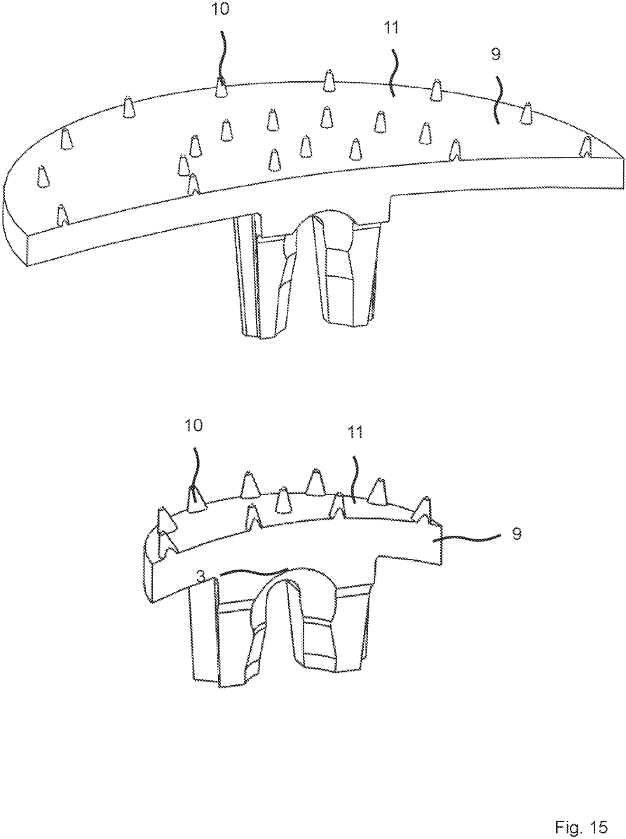

FIG. 15 a schematic perspective view of needle plates of different sizes with an arrangement of non-piercing stimulation needles with blunt needle tip in the area of a frontal application surface in section;

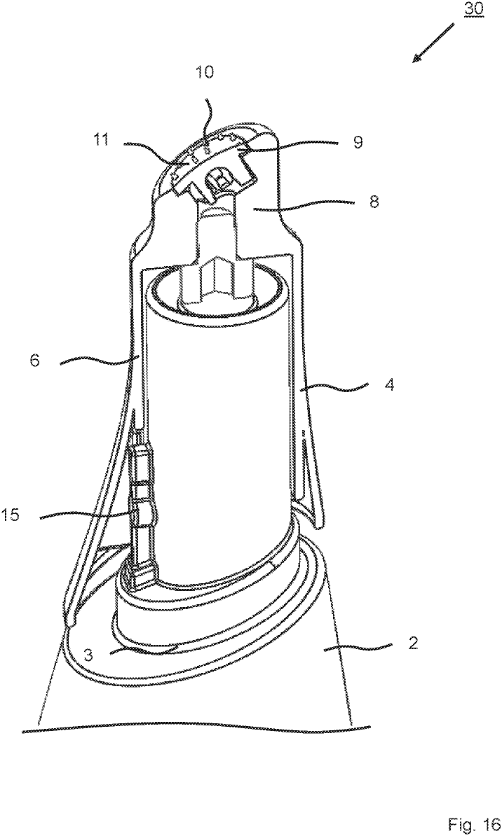

FIG. 16 a schematic perspective view of a front section of another handheld microneedling device partially in section;

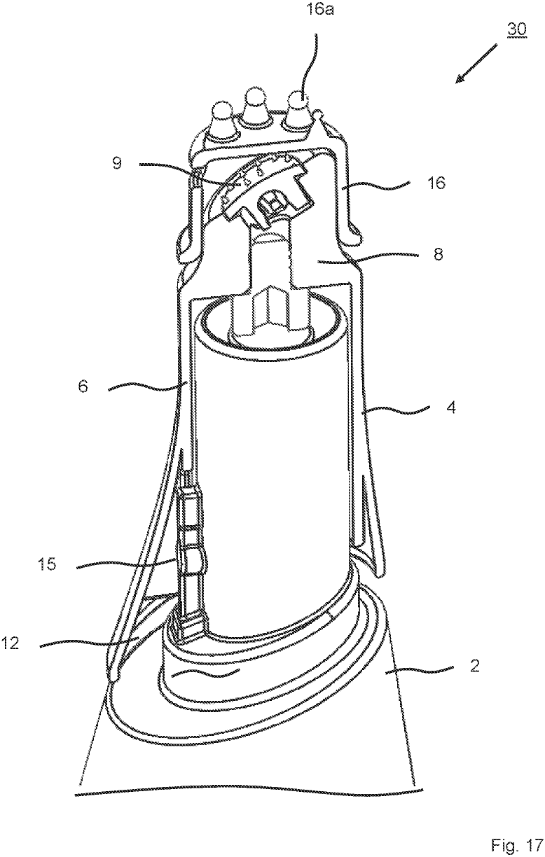

FIG. 17 a schematic perspective view of the front section of the further handheld microneedling device from FIG. 15 with selectively releasably attached cover or protective cap;

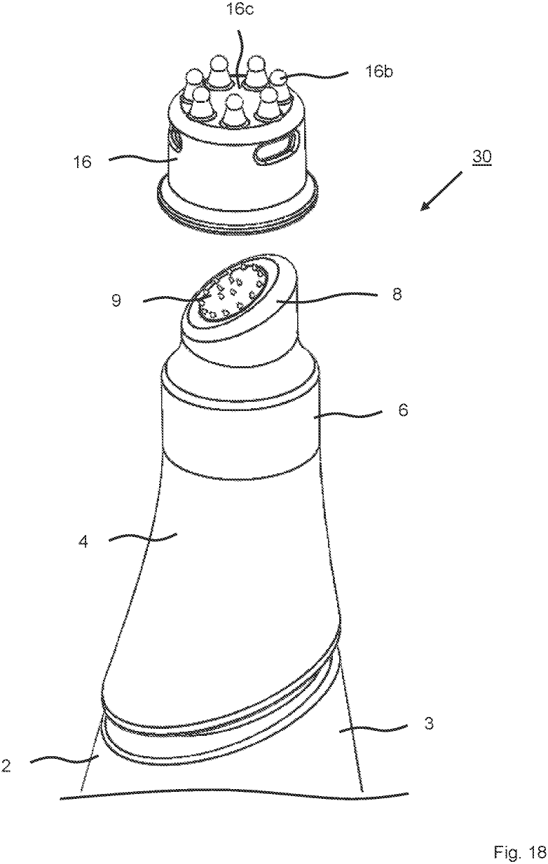

FIG. 18 a schematic perspective view of the front section of the further handheld microneedling device of FIG. 16 with the cover or protective cap removed, and

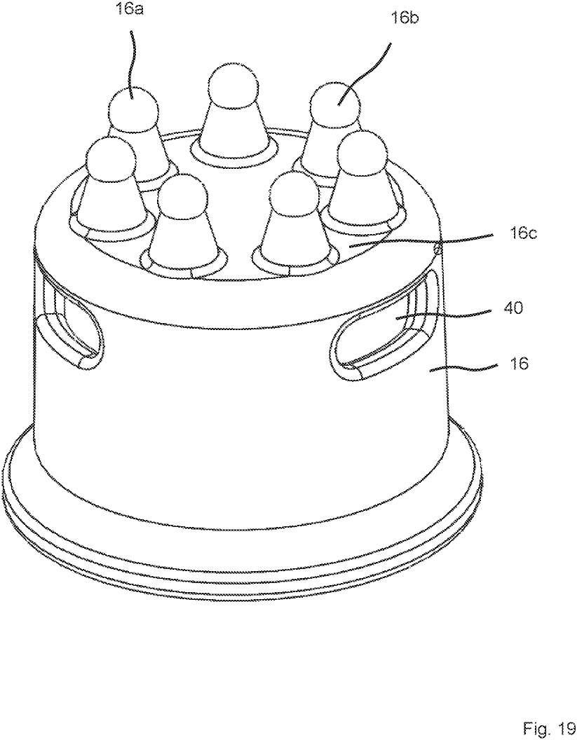

FIG. 19 a schematic perspective view of the cover or protective cap.

FIGS. 1 to 7 show perspective illustrations for a handheld microneedling device 1, which has a housing 2, which in the example shown is designed in several parts. The housing 2 is formed with a rear housing part 3 and a front housing part 4, which are associated with a drive module 5 and a skin stimulation device 6 selectively releasably arranged thereon, with which a stimulation module 7 is optionally formed. According to FIG. 2, the skin stimulation device 6 and thus optionally the stimulation module 7 is detachable from the drive module 5 and can be changed in this way. In the example shown, the drive module 5 and skin stimulation device 6 are selectively releasably connected with a plug-in connection.

In the rear housing part 3, which is associated with the drive module 5, a drive device is arranged, for example an electric motor, with which a drive movement or force is provided, which is coupled to a microneedling stimulation tool 8 of the skin stimulation device 6 in order to move the microneedling stimulation tool 8 back and forth in the longitudinal direction of the housing 2 during operation by means of the provided driving force with a repetition frequency. In this way, the microneedling stimulation tool 8, which is formed with a needle plate 9, is repetitively displaced between a front working position (FIG. 3) and a rear working position (FIG. 4) according to FIGS. 3 and 4. In this manner, during operation, a plurality of stimulation needles 10 protruding from the needle plate 9 in the region of an application surface 11 are locally applied (pressed on) to an associated skin portion in a non-piercing manner and withdrawn again. For this purpose, the several stimulation needles 10 are each designed with a needle tip that does not pierce the skin or is blunt (non-piercing).

In this case, the needle plate 9 with the plurality of stimulation needles 10 is exposed both in the front and in the rear working position according to FIGS. 3 and 4, i.e., in particular, it is not completely or partially surrounded by a section of the housing 2 and is thus arranged outside an inner space 12 of the housing 2. In this way, the local skin portion which comes into contact with the plurality of stimulation needles 10 can freely oscillate due to the back and forth movement of the microneedling stimulation tool 8 with the needle plate 9, which supports an efficient and preferably painless application on the skin.

In the example shown, the plurality of stimulation needles 10 are arranged in associated openings 13 in the region of the application surface 11 on the needle plate 9 and fixed therein, for example by means of bonding or soldering. In another embodiment, the plurality of stimulation needles 10 may be placed on the application surface 11, for example by means of welding. Alternatively, the stimulation needles 10 may be molded on by injection molding or integrated as insert components. The plurality of stimulation needles may all have substantially the same needle length or different needle lengths above the application surface 11, in which case groups of the plurality of stimulation needles 10 may be of the same needle length.

In the example shown in FIGS. 1 to 7, the skin stimulation device 6, thus optionally the stimulation module 7, is attached by means of coupling 14, which is formed with a coupling part 14a on the drive side and a coupling part 14b on the stimulation tool side. The coupling 14 is formed, for example, as a magnetic coupling between the coupling part 14a on the drive side and the coupling part 14b on the stimulation tool side (cf. FIG. 6). The coupling 14 is used to initiate the driving force/movement for moving the needle plate 9 back and forth with the stimulation needles 10.

In the example shown, an anti-rotation device 15 is provided to prevent the stimulation tool module 6 from rotating.

According to FIG. 5, the needle plate 9 with the multiple stimulation needles 10 can be covered in non-operation by means of a cover or protective cap 16, which is selectively releasably attached and thus removable for operation. In the example shown, the cover or protective cap 16 is attached.

FIGS. 6 and 7 show the front section of the handheld microneedling device 1 partially in section with and without the cover cap 16, revealing that the plurality of stimulation needles 10 extend in associated bores 17 of the needle plate 9 and are received at the rear of the needle plate 9 on a needle holder 18.

FIG. 8 shows a schematic perspective view of an electric toothbrush 19, in which a front housing part 19a is formed with a toothbrush head 19b, which is fitted in place of the skin stimulation device 6 and couples to the drive device in the rear housing part 3 of the housing 2, so that the driving force (back and forth movement) is used to move a brush arrangement 19c in operation. Skin stimulation device 6 and toothbrush head 19b can thus be selectively selectively releasably mounted and operated by one and the same drive module 5, so that either the handheld microneedling device 1 or the electric toothbrush 19 are provided.

FIGS. 9 to 13 show another handheld microneedling device 20 in which the microneedling stimulation tool 8 with the needle plate 9 is displaceable through a front housing opening 21 on the skin stimulation device 6 (stimulation module 7) between a retracted non-working position as shown in FIGS. 9 to 11 and extended working positions as shown in FIGS. 11 and 12.

When not in use, the needle plate 9 of the skin stimulation device 6 with the plurality of stimulation needles 10 arranged thereon can be retracted behind the surface of the housing opening 21 such that, according to FIG. 9, both the needle plate 9 and the stimulation needles 10 are arranged completely behind the housing opening 21 in the interior 12 of the front housing part 4, which in the embodiment shown is associated with the skin stimulation device 6. On the front housing part 4, the housing opening 21 is formed at a front section 4a.

During microneedling operation, the needle plate 9 with the multiple stimulation needles 10 is moved back and forth between the front working position (cf. FIG. 12) and the rear working position (cf. FIG. 13) as shown in FIGS. 12 and 13. 13), i.e. back and forth in the longitudinal direction of the housing 2 corresponding to a working stroke, so that the needle plate 9 with the plurality of stimulation needles 10 is arranged both in the front and in the rear working position with respect to the housing opening 21 in front of the surface thereof and thus outside the housing 2, in particular outside the front section 4a of the front housing part 4, which in turn enables the skin section to be processed to be stimulated to oscillate freely, i.e. free from any obstruction to this oscillation by the housing 2, in particular a front housing edge 22 surrounding the housing opening 21. In the example shown, in operation substantially the entire microneedling stimulation tool 8 is positioned in front of the housing opening 21. In non-operation, the needle plate 9 can then be displaced to the retracted non-working position as shown in FIGS. 9 to 10.

According to the example shown in FIGS. 9 to 13, the needle plate 9 is circumferentially and continuously spaced from the housing rim 22 when passing through the housing opening 21, i.e., it is free of contact and contact-free.

It may be provided that the needle plate 9 may assume several different retracted non-working positions within the front housing portion 4 behind the housing opening 21, which differ with respect to their respective distance from the housing opening.

In the area of the coupling 14, the rear stimulation tool-side coupling part 14b of the skin stimulation device 6 according to FIG. 10 is (selectively releasably) connected to the drive-side coupling part 14a, which is designed here as a shaft component, in order to couple the provided driving force/movement to the microneedling stimulation tool 8 with the needle plate 9.

FIG. 14 shows a schematic perspective view of an embodiment of the needle plate 9 with an arrangement of the plurality of non-piercing stimulation needles 10 on the application surface 11, each having a blunt needle tip 10a. In the shown embodiment, the plurality of stimulation needles 10 are formed with a cone shape and a flattened needle tip. The same reference numerals as in the preceding figures are used for identical features in FIGS. 14 to 18.

FIG. 15 shows a schematic perspective view of needle plates 9 of different sizes with an arrangement of non-piercing stimulation needles 10 with blunt needle tip in the area of the application surface 11 in section, whereby the application surface 11 is convexly curved.

FIGS. 16 to 19 show schematic perspective views of a front section of a further handheld microneedling device 30, in which the application surface 11 on the needle plate 9 of the microneedling stimulation tool 8 is set at an angle or inclined with respect to the longitudinal direction of the housing 2, thus in the example shown also at an angle or inclined with respect to the direction of movement when moving the microneedling stimu lation tool 8 (with the needle plate 9) between the front and rear working positions.

FIGS. 17 and 18 show the further handheld microneedling device 30 with the cover or protective cap 16 selectively releasably fitted and removed. A functional tool 16a is arranged on the cover cap 16, which in the fitted state (cf. FIG. 17) covers the plurality of non- piercing stimulation needles 10, which in the embodiment example shown is formed with an arrangement of functional elements 16b, which are for example massage elements. The cover cap 16 is thus configured as a multifunctional cap. In the illustrated embodiment example, the functional elements 16b are formed in particular in the region of a front surface 16c of the cover cap 16.

In addition, the cover cap 16 can have, at least on the outside, an inclined end face with functional tool. It is also conceivable that it can be folded away, or also an axially movable sleeve with a missing cover surface or a cover surface that is perforated for the functional tool (comparable to a type of drill bit with tools on the circumference or on the remaining cover surface), which can be switched on/off, e.g., by a rotary gate cam control via rotary-slide movement.

Other functional tools can be provided, for example silicone nubs, gripping-pinching tools made of elastic material and / or alternately directed stimulation tools, which in turn achieve a prick-like (simulating piercing) effect on the skin and thereby cause skin irritation. Alternative functional tools for skin stimulation can be provided, which differ from the aforementioned, for example, with regard to a tip radius or angle.

In one embodiment, the needle plate 9 can be designed as an reversible plate or reversible insert in such a way that different functional tools can be arranged on it in an exchangeable or detachable manner in order to provide several functions or to compensate for wear. They can also be interchangeable inserts that are selectively releasably arranged on the needle plate 9 depending on the desired application. Reversible inserts can carry tooling on both sides. The interchangeable inserts may be selectable from an assortment or graduated set of tool characteristics.

FIG. 19 shows an enlarged perspective view of the protective or cover cap 16 with the functional tool 16a. Openings 40 are provided on the cover cap 16. In the example shown, the openings are arranged in the region of a lateral surface 41 of the cover cap 16 and thus form lateral openings. Alternatively or additionally, such openings may be arranged in the region of the front surface 16c in other embodiments (cf. FIG. 5). For example, the openings 40 may serve to perform gas sterilization for the needle plate 9 with the plurality of stimulation needles 10 with the cover cap 16 in place. The lateral openings 40 shown in FIGS. 18 and 19 have the advantage that the ingress of dust particles to the needle plate 9 and dust deposits caused thereby are reduced.

The features disclosed in the foregoing description, the claims, and the drawing may be significant, both individually and in any combination, for the realization of the various embodiments.

1. A handheld microneedling device for local stimulation of a skin, with
a housing;
a drive device arranged in the housing and adapted to provide a driving force;
a skin stimulation device;
a microneedling stimulation tool formed on the skin stimulation device and connected to the driving device such that the microneedling stimulation tool is movable back and forth at a repetition frequency by the driving force when the driving force is in operation; and
a needle plate formed on the microneedling stimulation tool, and in which a plurality of non-piercing stimulation needles having a blunt needle tip are arranged distributed over a front-side application surface;
wherein the microneedling stimulation tool is movable back and forth by means of the driving force at the repetition frequency between a front working position and a rear working position which is set back in comparison thereto.

2. The handheld microneedling device according to claim 1, wherein, when moving between the front working position and the rear working position, at least the plurality of non-piercing stimulation needles of the needle plate in the front working position and the rear working position of the microneedling stimulation tool are arranged completely outside the housing and are thus exposed.

3. The handheld microneedling device according to claim 1, wherein, in operation, at least the front-side application surface is further arranged completely outside the housing and thus exposed both in the front working position and in the retracted rear working position of the microneedling stimulation tool.

4. The handheld microneedling device according to claim 1, wherein the needle plate is formed free of a housing portion of the housing at least partially surrounding the needle plate.

5. The handheld microneedling device according to claim 1, wherein the front-side application surface with the plurality of non-piercing stimulation needles distributed thereon, at least in the front working position, is inclined with respect to at least one reference direction from the following group: a longitudinal direction of the housing, a direction of movement of the microneedling stimulation tool when shifting between the front and the rear retracted working positions, and a direction of movement of the needle plate when shifting between the front and the rear retracted working positions.

6. The handheld microneedling device according to claim 1, wherein a protective cap which is selectively releasably arranged on the housing covers the needle plate, a functional tool being arranged on the protective cap on the outside.

7. The handheld microneedling device according to claim 1, wherein a front housing part, which is assigned to the skin stimulation device, is of sleeve-shaped design at the rear and at least partially encloses a rear housing part, which is assigned to the drive device.

8. The handheld microneedling device according to claim 1, wherein the skin stimulation device is selectively releasably arranged on the housing.

9. The handheld microneedling device according to claim 8, wherein the drive device is formed with a drive device for an electric toothbrush, which is configured to be selectively releasably coupled thereto the skin stimulation device and a toothbrush head operable by means of the drive device.

10. The handheld microneedling device according to claim 1, wherein the microneedling stimulation tool can be displaced through an opening at a front-side housing end of the housing into a non-working position which is further set back compared to the set-back rear working position and in which at least the front-side application surface of the needle plate is set back relative to the opening.

11. The handheld microneedling device according to claim 10, wherein the needle plate is spaced on one or more sides from an edge surrounding the opening during displacement through the opening.

12. The handheld microneedling device according to claim 10, wherein the needle plate in the further retracted non-working position is arranged to be exposed at a distance from housing portions of the housing surrounding the needle plate.

13. The handheld microneedling device according to claim 1, further comprising a reusable drive module in which the drive device is arranged, and a disposable module which is selectively releasably connected to the drive module and in which the skin stimulation device is arranged.

14. The handheld microneedling device according to claim 1, wherein the drive device is arranged to move the microneedling stimulation tool back and forth in operation at a repetition frequency between about 10 Hz and about 200 Hz by means of the driving force applied thereto.

15. A skin stimulation device having a housing and being couplable to a drive device for forming a handheld microneedling device according to claim 1, comprising:
a microneedling stimulation tool formed on the skin stimulation device and connectable to the drive device such that the microneedling stimulation tool is movable back and forth at a repetition frequency by the driving force provided by the drive device; and
a needle plate formed on the microneedling stimulation tool, and wherein a plurality of non-piercing stimulation needles having a blunt needle tip are arranged distributed over a front-side application surface;
wherein the microneedling stimulation tool is movable back and forth between a front working position and a rear working position which is set back in comparison thereto, and wherein at least the plurality of non-piercing stimulation needles of the needle plate are arranged completely outside the housing and thus exposed in the front working position and the rear working position of the microneedling stimulation tool.

16. A kit, comprising:
a drive device arranged in a housing;
a toothbrush head selectively releasably connectable to the housing and thus coupled to the drive device to define an electric toothbrush; and
a skin stimulation device which can be selectively releasably connectable to the housing and thus coupled to the drive device to define a microneedling device.
